(19) United States
(12) Patent Application Publication
(10) Pub. No.: US 2025/0090813 A1
(43) Pub. Date: Mar. 20, 2025

(54) GUIDEWIRE HAVING ENLARGED, MICRO-FABRICATED DISTAL SECTION

(71) Applicant: SCIENTIA VASCULAR, INC., West Valley City, UT (US)

(72) Inventors: John A. Lippert, Park City, UT (US); Edward J. Snyder, Park City, UT (US)

(21) Appl. No.: 18/961,143

(22) Filed: Nov. 26, 2024

Related U.S. Application Data
(63) Continuation of application No. 17/154,777, filed on Jan. 21, 2021, now Pat. No. 12,178,975.
(60) Provisional application No. 62/965,005, filed on Jan. 23, 2020.

(51) Int. Cl. A61M 25/09

(52) U.S. Cl. CPC A61M 25/09041; A61M 2025/09083; A61M 2025/09166

(57) ABSTRACT

Disclosed is a guidewire having a core wire and an outer tube within which the distal section of the core is inserted. The outer diameter of the tube is greater than the outer diameter of the proximal section of the core wire. The guidewire also includes a proximal coil and a distal, radiopaque coil each disposed over the distal section of the core. A bushing coil is disposed over the proximal coil and distal coil. The proximal coil, distal coil, and bushing coil aid in filling the annular space between the core and the tube and thereby centering and aligning the core and tube.

### CROSS-REFERENCE TO RELATED APPLICATIONS

This Application is a continuation of U.S. application Ser. No. 17/154,777, filed Jan. 21, 2021, which claims priority to and the benefit of U.S. Provisional Patent Application No. 62/965,005, filed Jan. 23, 2020. Each of the foregoing applications is incorporated herein by reference in its entirety.

### BACKGROUND

Guidewire devices are often used to lead or guide catheters or other interventional devices to a targeted anatomical location within a patient's body. Typically, guidewires are passed into and through a patient's vasculature in order to reach the target location, which may be at or near the patient's heart or brain, for example. Radiographic imaging is typically utilized to assist in navigating a guidewire to the targeted location. In many instances, a guidewire is placed within the body during the interventional procedure where it can be used to guide multiple catheters or other interventional devices to the targeted anatomical location.

Guidewires are available with various outer diameter sizes. Widely utilized sizes include 0.010, 0.014, 0.016, 0.018, 0.024, 0.035, and 0.038 inches, for example, though they may also be smaller or larger in diameter. Because torque transmission is a function of diameter, larger diameter guidewires typically have greater torque transmission (the ability to effectively transfer torque from proximal portions of the wire to more distal portions of the wire). On the other hand, smaller diameter guidewires typically have greater flexibility.

A catheter used in conjunction with a guidewire will be sized with an inner diameter somewhat larger than the outer diameter of the guidewire to enable the catheter to be positioned over and translated upon the guidewire. The difference in size between the guidewire and catheter can affect the ability of the catheter to travel along the guidewire. For example, the larger the annular space between the outer diameter of the guidewire and the inner diameter of the catheter, the greater the amount of potential radial offset the catheter may experience and the more difficult it may be to navigate the catheter over the guidewire. With excessive radial offset, the distal end of the catheter may have a higher risk of catching against vasculature or other anatomy of the patient rather than smoothly following along the guidewire path.

Often, a guidewire size is selected to minimize the amount of annular space between the guidewire and a given catheter size required or desired for a particular procedure, and to thereby limit the types of issues described above. However, several challenges exist to this approach. For example, increasing the size of the guidewire may overly increase the stiffness of the guidewire as well, potentially to levels that are undesirable for the initial placement of the guidewire at the targeted treatment site. Moreover, while there are known methods for increasing guidewire flexibility, such as reducing the core wire diameter, but these often come at the expense of torquability of the device.

What is needed, therefore, is a guidewire device capable of being manufactured with a relatively large outer diameter, at least at the distal section, that minimizes the annular space between the guidewire and certain sizes of compatible catheters, and that is also capable of providing sufficient flexibility and torquability along its length.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an embodiment of a guidewire device having a core and an outer tube and which may utilize one or more of the components described herein;

FIG. 2 illustrates an exemple embodiment of a guidewire device with a tube that has an outer diameter that is larger than an outer diameter of a proximal section of the core;

FIG. 3 is a detailed view of the distal section of the guidewire of FIG. 2, with the tube structure removed to better illustrate underlying features of the device;

FIG. 4 is a detailed view of the tube of the guidewire of FIG. 2;

FIG. 5 is a cross-sectional view of a distal section of the guidewire of FIG. 2, showing alignment of the beams of a one-beam section of the tube with a flattened distal section of the core; and

FIGS. 6 and 7 are cross-sectional views of the guidewire of FIG. 2 showing that the outer diameter of the tube is greater than the outer diameter of the proximal section of the core.

### DETAILED DESCRIPTION

### Introduction

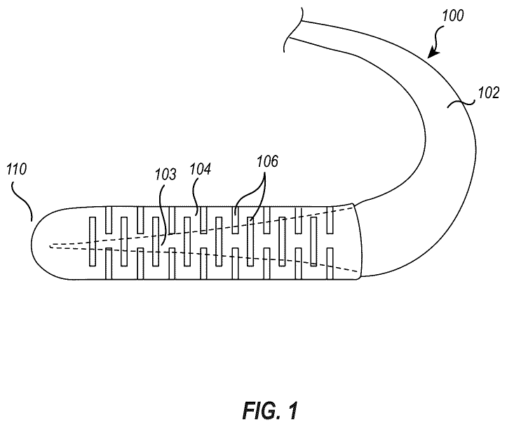

FIG. 1 schematically illustrates the general components of an intravascular device, shown here in the form of guidewire device 100, that may utilize one or more features described in greater detail below. The illustrated guidewire device 100 includes a core 102 and an outer tube 104. The core 102 includes a distal section (also referred to herein as the distal core 103) that extends into the outer tube 104 as shown. The distal core 103 may be tapered, either continuously or in one or more discrete sections, so that more distal sections have a smaller diameter and greater flexibility than more proximal sections. In some embodiments, the most distal section of the core 102 may be flattened into a ribbon-like shape with a flat, rectangular, or oblong cross section. For example, the distal core 103 may be ground so as to progressively taper to a smaller diameter at the distal end.

The core 102 and the tube 104 are typically formed from different materials. For example, the tube 104 is preferably formed from a relatively flexible and elastic material such as nitinol, whereas the core 102 may be formed from a relatively less flexible and elastic material such as stainless steel. Forming the core 102 from stainless steel (or other materials with similar modulus of elasticity) may be advantageous because it allows the distal tip to hold a shape when selectively bent/shaped by an operator and because stainless steel provides sufficient modulus of elasticity to provide more responsive translational movement. While these materials are presently preferred, other suitable materials such as polymers or other metals/alloys may additionally or alternatively be utilized.

The tube 104 is coupled to the core 102 (e.g., using adhesive, soldering, and/or welding) in a manner that beneficially allows torsional forces to be transmitted from the core 102 to the tube 104 and thereby to be further transmitted distally by the tube 104. A medical grade adhesive or other suitable material may be used to couple the tube 104 to the core 102 at the distal end 110 of the device to form an atraumatic covering.

The outer tube 104 may include a cut pattern that forms fenestrations 106 in the tube. The pattern of fenestrations 106 may be arranged to provide desired flexibility characteristics to the tube 104, including the promotion of preferred bending directions, the reduction or elimination of preferred bending directions, or gradient increases in flexibility along the longitudinal axis, for example. Examples of cut patterns and other guidewire device features that may be utilized in the guidewire devices described herein are provided in detail in United States Patent Application Publication Nos. 2018/0193607, 2018/0071496, and 2020/0121308, the entireties of each of which are incorporated herein by this reference.

The proximal section of the guidewire device 100 (the portion extending proximally from the tube 104) extends proximally to a length necessary to provide sufficient guidewire length for delivery to a targeted anatomical area. The guidewire device 100 typically has a length ranging from about 50 cm to about 350 cm, more commonly about 200 cm, depending on particular application needs. The tube 104 may have a length ranging from about 5 cm to about 350 cm, more typically about 15 cm to about 50 cm, such as about 25 cm to about 40 cm.

The guidewire device 100 may have a diameter of about 0.010 inches to about 0.038 inches, though larger or smaller sizes may also be utilized depending on particular application needs. For example, particular embodiments may have outer diameter sizes corresponding to standard guidewire sizes such as 0.014 inches, 0.016 inches, 0.018 inches, 0.024 inches, or other such sizes common to guidewire devices. The distal section of the core 102 (referred to herein as the distal core 103) may taper to a diameter of about 0.002 inches, or a diameter within a range of about 0.001 to 0.005 inches. In some embodiments, the distal tip may be flattened (e.g., to a rectangular cross section) to further enhance bending flexibility while minimizing reductions in cross-sectional area needed for tensile strength. In such embodiments, the cross section may have dimensions of about 0.001 inches by 0.003 inches, for example. In some embodiments, the tube 104 has a length within a range of about 3 to 350 cm.

Additional features and details regarding the foregoing components are described in further detail below. The following examples may be particularly beneficial in applications where the corresponding catheter is about sized at 0.027 inches or greater, and the guidewire is thus beneficially sized at about 0.024 inches or greater in order to limit the amount of annular space between the inner surface of the catheter and the outer surface of the guidewire, but still allow for relative movement between them. In such implementations, the guidewires described herein are able to provide sufficient diameter in the distal sections of the device to limit the annular space, while still maintaining effective torqueability and lateral flexibility. These sizes are not limiting, however, and the same features and details described below may also be utilized in guidewires that are smaller or larger than 0.024 inches.

### Improved Guidewire Device with Enlarged Distal Section

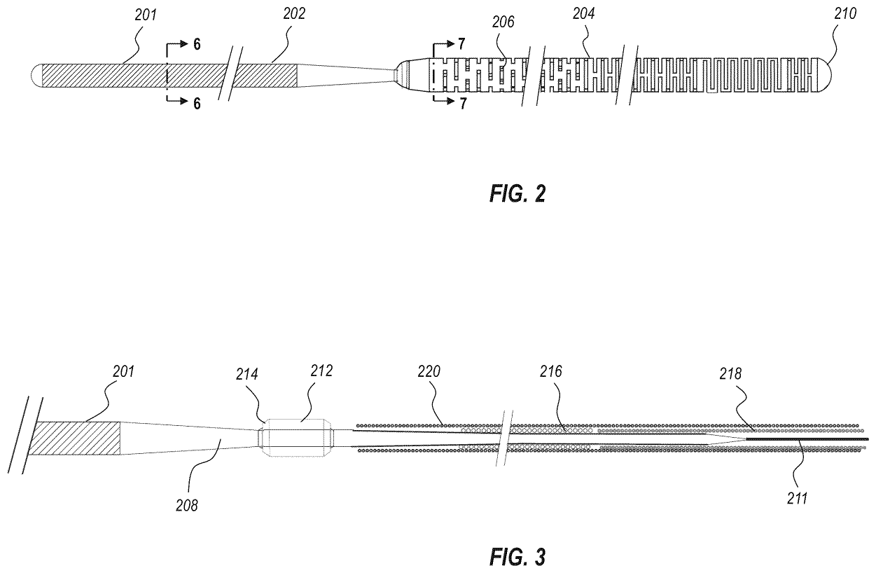

FIG. 2 illustrates an example of a guidewire 200. Except where noted herein, the guidewire 200 may include any of the general features described above in relation to guidewire 100, with like reference numbers indicating like parts. As shown, the guidewire 200 includes a core 202 and an outer tube 204, with a distal section of the core 202 (referred to as the distal core 203) inserted into the tube 204. The tube 204 includes a plurality of fenestrations 206. A polymer-based adhesive may form an atraumatic distal tip 210.

The core 202 also includes a proximal section (referred to herein as the proximal core 201) that is disposed proximal of the tube 204 and is not inserted into the tube 204. The proximal core 201 may comprise a friction-lowering coating, such as polytetrafluoroethylene (PTFE) and/or other suitable coating materials. The tube 204 may also include a coating, preferably a suitable hydrophilic coating and/or other suitable coating materials.

Preferably, the outer diameter of the tube 204 is slightly larger than the outer diameter of the proximal core 201. In one exemplary embodiment, the proximal core 201 has an outer diameter of about 0.018 inches, while the tube 204 has an outer diameter of about 0.024 inches. Other core and/or tube sizes may also be utilized, however. Preferably, the tube 204 has an outer diameter that is about 10% or more larger than the outer diameter of the proximal core 201, more preferably about 15% to about 80% larger, or more preferably about 20% to about 70% larger, such as about 25% to about 35% larger.

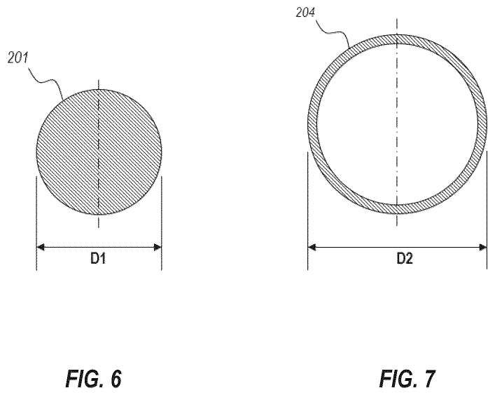

This is further illustrated by the cross-sectional views of FIGS. 6 and 7. As shown, the outer diameter (D1) of the proximal core 201 is less than the outer diameter (D2) of the tube 204. The ratio of D2 to D1 may be, for example, about 1.1 to about 3, more preferably about 1.15 to about 2, or about 1.2 to about 1.75.

As mentioned above, a larger outer diameter in the tube 204 can better match certain desired catheter sizes at the catheter distal tip portion and thereby reduce the amount of annular space between the guidewire and catheter during placement of the catheter over the guidewire. This is particularly beneficial at the more distal sections of the guidewire, which are more likely to be navigated through deeper, more tortuous portions of the patient's vasculature.

However, increasing the diameter of the core 202 to match the larger diameter of the tube 204 may make the core 202 too stiff for use in certain desired applications. Thus, maintaining a smaller core 202, while increasing the size of the tube 204 relative to the core 202, allows use of the more flexible core 202 while still enabling the benefits of a larger tube 204 at the distal sections of the guidewire 200.

As explained in more detail below, however, providing a tube 204 that has a larger outer diameter than the core 202 can introduce other challenges. In particular, the difference in diameter between the tube 204 and the distal core 203 enlarges the annular space between the outer surface of the distal core 203 and the inner surface of the tube 204. Because the tube 204 can be more flexible than the distal core 203, as the wire navigates a bend, the distal core 203 may be positioned off-center from the center line of the tube 204. As the guidewire is moved through the vasculature, this off-centering can disrupt the smooth distal transmission of rotational movement, causing a buildup and sudden release of forces which lead the guidewire to move with a “snap” and/or “whip” to a non-desired preferential rotational location. This disruption to the tactile feel and rotational control of the guidewire can make it more difficult for the operator to rotationally position the guidewire as intended, raising the risk of interventional procedure delays, suboptimal outcomes, inability to access the target location, or even tissue injury.

The embodiments described herein beneficially provide additional features that assist in radially centering the distal core 203 within the tube 204 even though the tube 204 has a larger outer diameter than the proximal core 201. One or more centering mechanisms may be included to beneficially reduce undesirable whip and/or snap movements of the guidewire (i.e., the centering mechanisms may improve rotational control), thereby enabling a user to have greater rotational control and improved tactile handling of the guidewire.

FIG. 3 illustrates an expanded view of the distal section of the guidewire 200 with the tube 204 removed in order to better visualize the distal core 203 and some of the other underlying components. As shown, the core 202 includes one or more transition zones 208 where the core 202 tapers to a smaller diameter. A distal end section 211 of the core 202 may be flattened. The one or more transition zones 208 may be discrete, with one or more sections of the core of substantially continuous outer diameter disposed between, or the distal core 203 may have a substantially continuous taper along all or most of its length.

A bushing 212 may be included at the point forming the joint to which the proximal end of the tube 204 is attached. The bushing 212 may have an outer diameter that substantially matches the outer diameter of the proximal core 201. The bushing 212 may be formed from the same material as the tube 204 (e.g., nitinol). The bushing 212 provides for better centering between the core 202 and the tube 204 and/or reduces the amount of adhesive needed to bond the separate components. Although shown here as a tube, the bushing 212 may have alternative geometries such as a coil, braid, slotted/cut tube, etcetera.

As shown, the bushing 212 may also include a chamfered or beveled surface 214 on its proximal end (i.e., proximal edge) to provide a smooth transition between different diameters. The distal end (i.e., distal edge) of the bushing 212 may also be chamfered or beveled. Even though the distal end of the bushing 212 will be covered by the tube 204, providing a bushing 212 with a chamfer/bevel on both ends can aid in manufacturing, eliminating the need to ensure proper orientation of the bushing and eliminating the potential for erroneous orientation.

The illustrated guidewire 200 includes a proximal coil 216, a distal coil 218, and a bushing coil 220 positioned over the proximal coil 216 and the distal coil 218. The distal coil 218 is preferably formed of a radiopaque material, such as platinum group, gold, silver, palladium, iridium, osmium, tantalum, tungsten, bismuth, dysprosium, gadolinium, and the like. The distal coil 218 thus preferably allows radiographic visualization of the distal end of the guidewire 200 during a procedure. The distal coil 218 may have a length of about 0.5 cm to about 20 cm, or more typically about 3 cm to about 15 cm, such as about 10 cm.

The proximal coil 216 may be formed from a non-radiopaque material such as stainless steel, other suitable metal, a suitable polymer, or other suitable material. The proximal coil 216 may be attached to the distal core 203 at a point adjacent to or near to the proximal end of the distal coil 218 and/or at any point along the coincident length of the distal core 203, most commonly at or near each end of the proximal coil 216. The proximal coil 216 may have a length of about 1 to 25 cm, or more typically about 3 to 20 cm, such as about 5 to 15 cm. Technically, the distal coil 218 could be extended further proximally to take the place of the proximal coil 216. However, materials that function well as radiopaque markers (e.g., platinum) are relatively expensive. Also, their use as a packing material to fill large portions of the annular space could cause the distal section of the guidewire 200 to be overly bright when imaged under x-ray fluoroscopy and thus not allow the operator to visualize other areas of interest. Thus, the proximal coil 216 is preferably separate from, and formed from a different material than, the distal coil 218.

The proximal coil 216 and the distal coil 218 aid in filling some of the annular space between the distal core 203 and the tube 204. Although the coil examples illustrated herein are shown having wires with a circular cross section, it will be understood that other coil types may also be utilized. For example, centering coil(s) may be edge-wound and/or may have a ribbon, rectangular, oblong, or other non-circular shaped cross-sectional shape.

Although the proximal coil 216 and distal coil 218 aid in filling some of the annular space, additional annular space remains, particularly when a somewhat larger tube 204 is utilized. The wire size of the proximal coil 216 and distal coil 218 could be increased to fill more space. However, increasing wire size too much may introduce excessive stiffness to the device. Preferably, the wire size of the proximal coil 216 and distal coil 218 is about 0.008 inches or less, or about 0.006 inches or less, or more preferably about 0.004 inches or less, such as about 0.002 inches or less.

To aid in filling the remainder of the annular space, the guidewire 200 may include a bushing coil 220. The bushing coil 220 may be disposed over the proximal coil 216 and the distal coil 218. The bushing coil 220 may extend over the entirety of both of the proximal coil 216 and the distal coil 218. As with the proximal coil 216 and the distal coil 218, the wire diameter of the bushing coil 220 is preferably limited. For example, the wire diameter of the bushing coil may be about 0.008 inches or less, or about 0.006 inches or less, or more preferably about 0.004 inches or less, such as about 0.002 inches or less. The bushing coil 220 may be formed of stainless steel and/or other suitable material, such as another metal or a polymer.

The use of a bushing coil 220 in addition to the proximal coil 216 and distal coil 218 aids in filling the annular space between the distal core 203 and the tube 204 without the use of over-sized coils. This aids in maintaining centering of the distal core 203 within the tube 204, which prevents the undesirable effects of misalignment that have been described above while also minimally impacting the bending flexibility of the device.

In some embodiments, the bushing coil 220 may be substantially coincident with the proximal coil 216 and the distal coil 218. Alternatively, as shown, the bushing coil 220 may extend farther proximally than the proximal coil 216. This allows the bushing coil 220 to fill in more of the annular space even at portions where the proximal coil 216 is not able. That is, because of the tapered profile of the distal core 203, certain more proximal portions of the annular space do not fit both the proximal coil 216 and the bushing coil 220, but may still be filled by the further extending proximal portion of the bushing coil 220. The bushing coil 220 preferably extends along a substantial portion of the length of the tube 204. For example, the bushing coil 220 may have a length of at least about 60% of the length of the tube 204, or at least about 75% of the length of the tube 204, or at least about 80% of the length of the tube 204, or at least about 85% of the length of the tube 204.

In preferred embodiments, the proximal coil 216 and the distal coil 218 are each wound in a first direction, while the bushing coil 220 is counter-wound in a second, opposite direction. This beneficially limits interlocking and binding of the bushing coil 220 to either of the proximal coil 216 or the distal coil 218. The bushing coil 220 may also have a pitch that is different (e.g., narrower) than that of the proximal coil 216 or the distal coil 218. For example, the proximal coil 216 and/or the distal coil 218 may have a pitch of about 0.002 inches to about 0.008 inches, or about 0.003 inches to about 0.007 inches, whereas the bushing coil 220 may have a pitch of about 0.001 inches to about 0.006 inches, or about 0.002 inches to about 0.005 inches.

The proximal coil 216, the distal coil 218, and the bushing coil 220 are preferably configured to fill a substantial portion of the volume of the annular space between the distal core 203 and the tube 204. For example, proximal coil 216, the distal coil 218, and the bushing coil 220 may be configured to fill approximately 20% or more, 35% or more, 50% or more, 60% or more, 70% or more, 80% or more, or even up to about 90% or more of the volume of the annular space. Of course, other conventional guidewires may include joints or bushings that fill up large portions of the annular space at the particular part of the guidewire they are located. However, when the entire length of the outer tube is considered, such joints and bushings fill relatively little of the volume of the overall annular space.

The centering mechanism principles described herein may be utilized with other structural configurations to provide beneficial centering effects. For example, while the above embodiments describe various centering mechanisms with a core as the “inner member” and a microfabricated tube as the “outer member,” other structures may additionally or alternatively be utilized as the outer and/or inner members along with one or more of the described centering mechanisms.

For example, the inner member may be a wire (such as a ground core as described above), a tube (e.g., metal or polymer hypotube or metal or polymer microfabricated tube), a braid, or a coil. By way of further example, the outer member may be a tube (e.g., metal or polymer hypotube or metal or polymer microfabricated tube), a braid, a coil, or a polymer tube impregnated with a braid or coil. The centering mechanism may include a set of coils such as those described above, or may additionally or alternatively include other structures for providing centering of the inner member within the outer member. For example, one or more of the coils 216, 218, 220 may be replaced by one or more tubes (e.g., metal or polymer hypotube or metal or polymer microfabricated tube), braided sections, or sets of stacked rings.

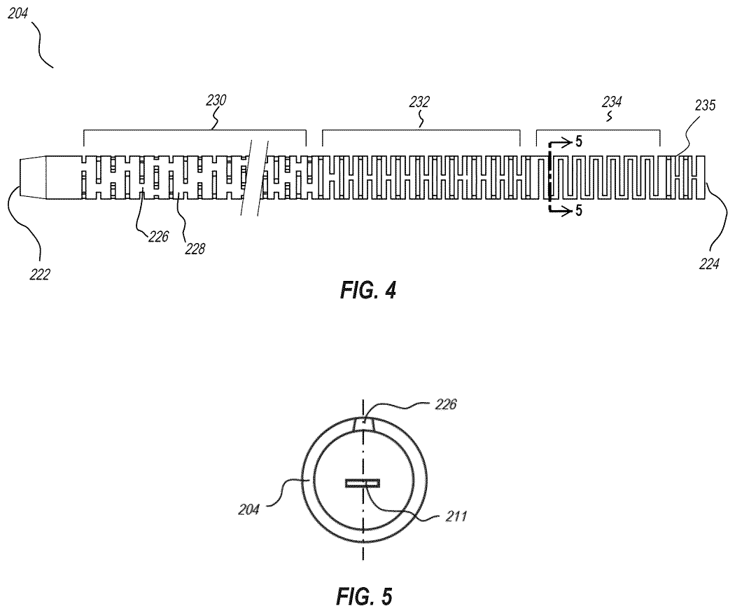

FIG. 4 illustrates the tube 204 separate from the core 202 and some of the other components of the device. The tube 204 extends between a proximal end 222 and a distal end 224. The fenestrations 206 formed within the tube 204 may be made according to a variety of cut patterns. Preferably, the overall effect of the fenestrations provides a flexibility gradient across the tube 204 where more flexibility increases closer to the distal end 224. Typically, greater flexibility can be provided by removing more of the stock material, such as by increasing the depth of the cut, decreasing the space between adjacent cuts, and/or reducing the number of axially extending beams 226 connecting each of the circumferentially extending rings 228.

The illustrated embodiment, for example, may include a three-beam section 230 (three beams connecting each adjacent pair of rings) that transitions to a two-beam section 232 (two beams connecting each adjacent pair of rings) that transitions to a one-beam section 234 (a single beam connecting each adjacent pair of rings). Within each of these sections, the cut depth and/or cut spacing may also be adjusted to provide a smooth intra-section and inter-section flexibility gradient. For example, the two-beam section 232 may have progressively less distance between cuts as it advances toward the distal end 224. It may then transition to the one-beam section 234, which itself then includes progressively less distance between cuts as it advances toward the distal end 224.

The one-beam section 234 may have a length of about 0.5 cm to about 3 cm, or about 0.75 cm to about 2 cm, for example. The two-beam section 232 may have a length of about 4 cm to about 16 cm, or about 6 cm to about 12 cm, for example. The three-beam section 230 may have a length of about 12 cm to about 36 cm, or about 18 cm to about 30 cm, for example. In other words, the three-beam section 230 may be about 2 to 5 times longer than the two-beam section 232, and the two-beam section 232 may be about 2 to 5 times longer than the one-beam section 234. Designing the tube 204 with these proportions of cut/beam sections has been found to provide effective balance of axial, lateral, and torsional stiffness for most applications.

The tube 204 may also include a distal-most section 235 that has a two-beam pattern. This section is preferably relatively short, such as about 0.5 cm or less, or about 0.25 cm or less, or about 0.15 cm or less. Providing a relatively short two-beam section at section 235 provides added surface area for an adhesive material applied at or near the distal end 224 of the tube 204 to bond, allowing a stronger coupling between the distal end 224 and any internal components bonded thereto.

Certain sections of the tube 204 may have cuts that are rotationally offset so as to avoid the formation of any preferred bending planes. For example, an angular offset may be applied after each cut or series of cuts such that the overall resulting pattern of beams 226 in the tube 204 do not align in a way that forms preferred bending planes.

Other sections of the tube 204 may include a preferred bending plane. For example, the one-beam section 234 may be aligned as shown in FIG. 4, with each beam offset by about 180° from the previous beam. These beams may also be aligned with the bending plane of the flattened distal end section 211 of the core. FIG. 5 illustrates, in cross-section, how the beams 226 of the one-beam section 234 are preferably aligned in the same plane as the flattened, wider section of the distal end section 211 of the core.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and the distal section of the core disposed within the outer tube;
a distal coil encompassing part of the distal section of the core;
a proximal coil disposed proximal of the distal coil and encompassing part of the distal section of the core; and
a bushing coil disposed over at least a portion of one or both of the distal coil and the proximal coil,
wherein the distal coil, proximal coil, and bushing coil fill at least a portion of the annular space, and
wherein the outer tube includes fenestrations that form a plurality of axially extending beams connecting circumferentially extending rings, wherein the fenestrations form a two-beam section proximal of a one-beam section proximal of a distal-most two-beam section.

2. The device of claim 1, wherein the distal coil is more radiopaque than stainless steel and the proximal coil is less radiopaque than the distal coil.

3. The device of claim 1, wherein the bushing coil extends farther proximally than the proximal coil.

4. The device of claim 1, wherein the bushing coil has a length of at least about 60% of the length of the tube.

5. The device of claim 1, wherein at least one of the proximal coil, distal coil, and bushing coil is wound in a direction opposite the other coils.

6. The device of claim 5, wherein the proximal coil and the distal coil are each wound in a first direction, while the bushing coil is counter-wound in a second, opposite direction.

7. The device of claim 1, wherein the bushing coil has a pitch that is smaller than that of the proximal coil and/or distal coil.

8. The device of claim 1, wherein an outer diameter of the outer tube is larger than an outer diameter of the proximal section of the core by 10% or greater.

9. The device of claim 1, further comprising a bushing disposed at a proximal end of the outer tube to aid in coupling the outer tube to the core, wherein a proximal edge and/or distal edge of the bushing is chamfered or beveled.

10. The device of claim 1, wherein a flattened distal section of the core has a preferred bending plane and wherein the preferred bending plane of the flattened distal section is aligned with a preferred bending plane of a portion of the tube overlying the flattened distal section.

11. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and the distal section of the core disposed within the outer tube;
a distal coil encompassing part of the distal section of the core;
a proximal coil disposed proximal of the distal coil and encompassing part of the distal section of the core; and
a bushing coil disposed over at least a portion of one or both of the distal coil and the proximal coil,
wherein the bushing coil has a length of at least about 60% of the length of the tube,
wherein the proximal coil and the distal coil are each wound in a first direction, while the bushing coil is counter-wound in a second, opposite direction, and
wherein the bushing coil has a pitch that is smaller than that of the proximal coil and/or distal coil.

12. The device of claim 11, wherein the bushing coil extends farther proximally than the proximal coil.

13. The device of claim 11, wherein the distal coil is more radiopaque than stainless steel and the proximal coil is less radiopaque than the distal coil.

14. The device of claim 11, wherein an outer diameter of the outer tube is larger than an outer diameter of the proximal section of the core by 10% or greater.

15. The device of claim 11, wherein a flattened distal section of the core has a preferred bending plane and wherein the preferred bending plane of the flattened distal section is aligned with a preferred bending plane of a portion of the tube overlying the flattened distal section.

16. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and an outer surface of the distal section of the core;
one or more coils disposed between the inner surface of the outer tube and the outer surface of the distal section of the core, the one or more coils filling at least a portion of the annular space; and
a bushing disposed at a proximal end of the outer tube to aid in coupling the outer tube to the core,
wherein the bushing comprises a chamfered or beveled proximal edge and a chamfered or beveled distal edge.

17. The device of claim 16, wherein a flattened distal section of the core has a preferred bending plane and wherein the preferred bending plane of the flattened distal section is aligned with a preferred bending plane of a portion of the tube overlying the flattened distal section.

18. The device of claim 16, wherein the one or more coils comprise: a distal coil encompassing part of the distal section of the core; a proximal coil disposed proximal of the distal coil and encompassing part of the distal section of the core; and a bushing coil disposed over at least a portion of one or both of the distal coil and the proximal coil.

19. The device of claim 18, wherein the distal coil is more radiopaque than stainless steel and the proximal coil is less radiopaque than the distal coil.

20. The device of claim 18, wherein the bushing coil has a pitch that is smaller than that of the proximal coil and/or distal coil.
